(19) United States
(12) Patent Application Publication
(10) Pub. No.: US 2026/0131092 A1
(43) Pub. Date: May 14, 2026

(54) ANESTHESIA SYSTEM AND PROCESS FOR CONTROLLING GAS QUANTITIES IN AN ANESTHESIA SYSTEM

(71) Applicant: Drägerwerk AG & Co. KGaA, Lübeck (DE)

(72) Inventors: Andreas KATER, Lübeck (DE); Björn BARGSTÄDT, Lübeck (DE)

(21) Appl. No.: 19/381,087

(22) Filed: Nov. 6, 2025

(30) Foreign Application Priority Data
Nov. 12, 2024 (DE) 10 2024 133 027.9

(51) Int. Cl. A61M 16/01; A61M 16/00; A61M 16/08; A61M 16/10

(52) U.S. Cl. CPC A61M 16/01; A61M 16/0003; A61M 16/024; A61M 16/0891; A61M 16/1005; A61M 2016/1025; A61M 2202/0007; A61M 2202/0208; A61M 2202/0241; A61M 2205/3327; A61M 2205/502

(57) ABSTRACT

A process controls gas quantities in a dosing and supply of quantities of oxygen for an anesthesia system. In a sequence of steps (101, 102, 103), a verification is carried out to determine whether there is a reason (11) for a reduction in the quantity of oxygen provided in the breathing gas and, depending on the result of the verification, the supply of quantities of oxygen is limited.

### CROSS REFERENCE TO RELATED APPLICATIONS

This application claims the benefit of priority under 35 U.S.C. § 119 of German Application 1 10 2024 133 027.9, filed Nov. 12, 2024, the entire contents of which are incorporated herein by reference.

### TECHNICAL FIELD

The present invention pertains to a process for controlling gas quantities when dosing and supplying quantities of oxygen to an anesthesia system and pertains to an anesthesia system. Anesthesia systems are used to safely administer inhalation anesthesia. Modern anesthesia systems have a closed or semi-closed breathing system, often also referred to as a circuit system, in which most of the breathing gas does not leave the device. The exhaled carbon dioxide is absorbed by a CO₂ absorber (breathing lime—soda lime), and fresh gas is added to the exhaled gas when it is returned to the circuit. This process has the advantage that the substances used for anesthesia (general anesthetics) can be used efficiently.

### BACKGROUND

Different variants of anesthesia devices with a radial blower (blower, radial compressor, fan) are described in U.S. Pat. No. 5,875,783 A. U.S. Pat. No. 5,875,783 A shows a circuit system that can be configured with a radial blower. For the purpose of understanding the function and advantages of the circuit system according to this prior art, as described in U.S. Pat. No. 5,875,783 A, the functions of the circuit system are described in this application with reference to the description of the figures (in FIG. 6 of U.S. Pat. No. 5,875,783 A). During inhalation, a radial blower draws in an anesthetic gas, formed as a mixture of oxygen, air, nitrous oxide and vaporized anesthetic agent, from a so-called fresh gas line and also buffered breathing gas from a manual ventilation bag as inhalation gas. If the pressure level in the patient's lungs is lower than the pressure level at the radial blower, this inhalation gas passes through a carbon dioxide absorber and then through an inspiratory non-return valve (check valve) via breathing tubes, a patient connecting element (patient Y-piece) and breathing access (breathing mask, endotracheal tube, tracheostoma) to and into the patient. As soon as the pressure conditions are reversed, i.e. as soon as the pressure level in the patient's lungs is above the pressure level at the radial blower, the gas flows from the patient through an expiratory non-return valve back into the manual ventilation bag.

When supplying gases, gas mixtures containing oxygen and anesthetic gases, it is necessary to design a resource-saving management of gas quantities during anesthesia in order to avoid wasting valuable gas resources of oxygen and for reasons of climate protection with regard to the climate-damaging effect of anesthetic gases such as desflurane, isoflurane, enflurane, sevoflurane and halothane.

Whenever concentrations of oxygen or anesthetic gas are changed during the course of anesthesia, the question must therefore be asked as to how quickly a gas change must take effect on the patient. If the user initiates flooding with 100% oxygen—for example before intubating the patient—this can be done without a significant time delay if the anesthesia device's O₂ flush functionality is used. The gas mixture previously present in the anesthesia device's line system is flushed out into the anesthetic gas scavenging system and a high concentration of oxygen is immediately present in the line system and in the breathing gas mixture for delivery to the patient.

However, high concentrations of oxygen over a longer period of time can pose a health risk to patients, which is described as “oxygen toxicity”. Prolonged exposure to a high inspiratory oxygen fraction (FiO₂), for example, can lead to damage to the alveoli, which is known as pulmonary oxygen toxicosis.

Delayed effects can be pulmonary edema or pulmonary fibrosis. This can result in effects on the nervous system with dizziness or nausea, for example. Oxygen concentrations above 60% can lead to irreversible damage to the eyes, particularly in newborns.

Therefore, different situations with high concentrations of oxygen can arise during anesthesia, which the user—usually a specialist in anesthesia or intensive care medicine—would like to reduce as quickly as possible over time for the therapeutic and/or physiological reasons mentioned above.

In a control loop-based anesthesia system in a conventional design according to the state of the art, a reduction in the oxygen setpoint usually results in the newly set reduced oxygen setpoint being adjusted by setting the minimum oxygen concentration of 21% for the fresh gas mixture (FG) of medical air (air), oxygen, possibly nitrous oxide and anesthetic agent, the amount of fresh gas mixture (FG) is increased and thus the reduction of the actual oxygen value in the breathing gas mixture for the patient is brought about by a “wash out” from the circuit system into the anesthetic gas scavenging system (NGF). During the period with the increased quantity of fresh gas (FG), increased quantities of anesthetic agent are also removed from the circuit system by means of the “wash out” in order to maintain a constant concentration of anesthetic agent in the breathing gas mixture for the patient.

### SUMMARY

Based on the state of the art, it is an object of the invention to further develop a process for controlling gas quantities in an anesthesia system and an anesthesia system in such a way as to achieve an improvement in the case of induced reductions in quantities of oxygen

The object is attained, and the problem is solved by features according to this disclosure.

The object is attained, and the problem is by a process for controlling gas quantities in an anesthesia system and by an anesthetic device with features of this disclosure.

The present invention provides a way to overcome the disadvantages of the prior art and to provide improved control of gas quantities—in particular oxygen—in the anesthesia system both in terms of patient comfort, in terms of operating aspects and in terms of environmental aspects.

Embodiments provide possibilities for developing the process for controlling gas quantities in a dosage and supplying quantities of oxygen to an anesthesia system as well as an anesthesia system. Further features and details of the invention and advantageous embodiments are presented in the claims, the description and the drawings.

Furthermore, with regard to an interpretation of the claims and the description, if a feature is specified in more detail, it must be assumed that such a limitation is not present in the respective preceding claims or in a more general embodiment of the device in question.

Any reference in the description to particular aspects is therefore to be read expressly as a description of optional features, even without specific reference.

First, in addition to the aforementioned prior art, an anesthesia system which forms the basis for implementing the process according to the invention is to be briefly outlined in order to understand the anesthesia environment. Such an anesthesia system for performing anesthesia on a living being has at least the following components:
a control unit
a breathing gas supply system with a mixing unit, a circuit system, an absorber unit for removing carbon dioxide (CO₂) from the circuit system and a gas conveying unit for conveying quantities of a gas mixture for performing anesthesia on the living being
a sensor system with
   at least one pressure sensor P1
   at least one gas sensor G1
   at least one flow sensor V1.

The mixing unit is configured to mix at least two gases to form a fresh gas mixture FG and is configured to provide this as a breathing gas mixture.

The gas conveying unit can, for example, be configured as a blower drive with a radial blower or as a piston drive with a piston and is configured to convey quantities of the breathing gas mixture. The gas conveying unit is configured and intended to deliver the gas mixture to the patient.

The breathing gas supply system has means—for example the gas conveying unit and/or in particular dosing valves or controllable proportional valves—for a controlled supply and/or dosing of an inspiratory breathing gas quantity and a device for controlling an expiratory breathing gas quantity—for example and in particular an expiratory valve (PEEP valve—Positive End-Expiratory Pressure valve)—for controlling an expiratory breathing gas quantity.

The breathing gas supply system also has a mixing unit with an oxygen dosing unit for controlled additional dosing of quantities of oxygen into the circuit system. The circuit system pneumatically connects the components of the breathing gas supply system, components of the sensor system and other components with each other and provides an inspiratory connection and an expiratory connection to form pneumatic connections with the line system.

With the mixing unit, the circuit system enables gases to be mixed to form a gas mixture that is suitable and intended for anesthesia and can be provided to a patient by the circuit system. In addition to air and oxygen, the gas mixture as so-called “fresh gas” also optionally consists of nitrous oxide and usually a volatile anesthetic agent (halothane, desflurane, enflurane, sevoflurane, isoflurane), which is introduced into the gas mixture by the anesthetic dosing unit, for example in the form of a so-called vaporizer.

In possible variants or configurations of anesthesia systems or anesthesia devices, a breathing bag can be arranged on the circuit system or on the breathing gas supply system. Such a breathing bag can be used for manual ventilation or hand ventilation by the user—for example during certain phases of a surgical procedure. A valve (APL valve—adjustable pressure limitation) can be used to limit the airway pressure supplied to the patient. Possible suitable positions for the breathing bag (BB) are on the breathing gas supply system or on the expiratory path of the circuit system.

In conventional embodiments, the anesthesia system can have further components for carrying out inhalation anesthesia and/or intravenously administered anesthesia, such as
an anesthetic dosing unit
an anesthetic gas scavenging system
a flush valve (purge valve/rinse valve) assembly
an APL valve assembly
a breathing bag
further sensors for pressure and flow rate measurement or for gas measurement

The breathing bag is a reservoir in the circuit system that absorbs the quantities of breathing gas mixture exhaled by the patient.

The flush valve arrangement can be configured as a controllable dosing valve and is configured to flush parts or components of the circuit system or the entire circuit system.

The APL valve arrangement provides an adjustable pressure limitation valve (APL valve) in the circuit system In a simplified version of the circuit system, the breathing bag, flush valve and/or APL valve arrangement can be omitted.

During operation, the anesthesia system is supplemented by a line system with the following components
a patient connecting element (Y-piece)
an inspiratory breathing tube
an expiratory breathing tube.

The line system is configured as a breathing tube system (ventilation tube system) with the connecting element (Y-piece) for supplying breathing gas quantities (inhalation gas) to the living being and for guiding breathing gas quantities (exhalation gas) away from the living being and thus serves to pneumatically and fluidically connect the patient to the circuit system of the anesthesia system. An inspiratory non-return valve is arranged in the inspiratory path of the line system and an expiratory non-return valve is arranged in the expiratory path in order to clearly define a direction of flow of inhaled and exhaled gas quantities in the circuit system and in the line system. For this purpose, the breathing tubes are connected to the circuit system on the device side with inspiratory and expiratory connections and connected to the patient connection element on the patient side. An element (patient interface) for supplying gas to the patient, such as an endotracheal tube, a face mask or a tracheostoma (tracheal access), is connected to the patient connection element.

The at least one flow sensor V1 is arranged on the breathing gas supply system, on the circuit system or on the line system in such a way that it continuously detects at least one flow rate which can indicate quantities of breathing gas mixture supplied to the living being or quantities of breathing gas mixture carried away by the patient, or can indicate quantities of breathing gas mixture supplied or carried away over a period of time and provide the control unit with measured values.

The at least one gas sensor G1 in the form of an oxygen sensor is arranged on the breathing gas supply system, on the circuit system or on the line system in such a way as to continuously detect at least one gas concentration which indicates a current concentration of oxygen supplied to the living organism or to indicate a time course of current quantities of oxygen supplied and to provide the control unit with the measured values.

The at least one pressure sensor P1 is arranged on the breathing gas supply system, on the circuit system or on the line system in such a way that it continuously records measured values that indicate at least one pressure level and provides these measured values to the control unit. The measured values indicate an airway pressure or a time course of an airway pressure.

In possible variants or configurations of anesthesia systems or anesthesia devices, an anesthetic gas scavenging device may be present or connected to the circuit system. Such an anesthetic gas scavenging device is used to quickly release a current gas mixture or used gas quantities from the circuit system. A possible suitable position for connecting an anesthetic gas scavenging device (NGF, AGS) is, for example, on the expiratory path on the circuit system.

Quantities of gas mixture are delivered to the patient in the circuit system via the inspiratory path, in which an inspiratory non-return valve is located, which prevents gases from flowing back from the patient into the inspiratory path.

The return flow from the patient takes place via the expiratory path into the circuit system.

An expiratory non-return valve is located in the expiratory path, which prevents gases from flowing back to the patient. Gas is supplied to the patient by means of the patient connection element, at which the inspiratory path is brought together and connected to an inspiratory breathing tube and the expiratory path to an expiratory breathing tube. During automatic ventilation, the gas conveying unit delivers a breathing gas mixture from the mixing unit through the circuit system into the inspiratory path as inspiratory gas to the patient during the inspiratory phase. During ventilation, the expiratory gas flows from the patient through the expiratory non-return valve during the expiratory phase back into the circuit system.

The control unit is configured and intended to organize, monitor, control or regulate the operation and/or sequence of the anesthesia system. The control unit is preferably made up of components (μC, μP, PC) with associated operating system (OS), data memory (RAM, ROM, EEPROM) and software (SW) code, software for sequence control, monitoring, control and regulation. In at least some embodiments, further electronic elements such as components for signal acquisition (ADμC), signal amplification, analog and/or digital signal processing (ASIC), components for analog and/or digital signal filtering (DSP, FPGA, GAL, μC, μP), signal conversion (A/D converter) are assigned to the control unit or connected to the control unit. The control unit is configured to carry out a control and coordination of inspiratory and expiratory quantities of breathing gas mixture for ventilation of the living being on the basis of the measured values provided by the sensor system with the breathing gas supply system, in particular with the means for controlled dosing and the device (PEEP valve) for controlling the expiratory breathing gas quantity. An anesthetic dosing unit can be used to dose anesthetic gases into the inspiratory and expiratory quantities of respiratory gas mixture, which can be controlled and coordinated by the control unit, thus enabling anesthesia or inhalation anesthesia to be administered to the living being. Measured values of the at least one pressure sensor P1 and or measured values of the at least one flow rate sensor can be taken into account by the control unit to control the timing of inspiration and expiration. Based on the measured values of the at least one pressure sensor P1 and/or the at least one flow rate sensor, the control unit can determine the breathing phases with the sequence of inspiration phases and expiration phases even when the patient is breathing spontaneously. Taking into account the measured values of the at least one flow sensor V1 and the measured values of the at least one pressure sensor P1, the control unit can thus determine the quantities of respiratory gas mixture supplied to the patient and the pressure levels thus given in inspiration (Pᵢₙₛₚ) and expiration (PEEP) and the sequence of inspiration and expiration, control, i.e. adjust, control or regulate the form of ventilation by controlling the gas conveying unit, for example by varying the speed of the radial blower of the blower drive or by changing the path of the piston of the piston drive accordingly, i.e. adjust, control or regulate. During operation of the anesthesia system, the control unit continuously records the measured values of the at least one pressure sensor P1 and the at least one flow sensor V1 with subsequent evaluation.

The control unit is configured to determine the volumes of breathing gas mixture currently supplied to the living being, such as tidal volume VT or minute volume MV, on the basis of the measured values of the at least one flow sensor V1. The control unit is also configured to determine the concentration of oxygen in the breathing gas mixture currently supplied to the living being on the basis of the measured values of the at least one gas sensor G1. The control unit is also configured to determine whether a situation exists in which there is a reason to reduce the amount of oxygen currently present in the line system or in the breathing gas. In particular, measured values from the at least one gas sensor provided by the sensor system are used by the control unit for this purpose and compared with reference values. Such a situation is characterized by a condition in which the current oxygen concentration in the respiratory gas supplied to the living being is above a predetermined upper oxygen concentration threshold value. The control of the process for controlling gas quantities when dosing and supplying quantities of oxygen to an anesthesia system is preferably carried out by means of a control unit in cooperation with a unit for mixing and dosing a gas mixture of at least two gas components in a line system and with a gas sensor G1, which is configured as an oxygen sensor.

The determination of the situation as to whether there is a reason (a basis) for a reduction in an amount of oxygen supplied to a patient or to a living being constitutes a start situation for carrying out the process for controlling gas quantities in a dosing and supply of quantities of oxygen to an anesthesia system.

In a process according to the invention for controlling gas quantities
in a first step, a verification (a check/a determination) is carried out (executed) to determine whether there is any reason to reduce the amount of oxygen currently present in the line system or in the breathing gas,
in a second step, if there is a reason to reduce the amount of oxygen currently present in the line system, an output signal is generated which indicates this reason and is transmitted to a unit for mixing and dosing a gas mixture,
in a third step, the amount of oxygen fed (supplied) into the line system is thus limited.

The process according to the invention with the embodiments described can also be referred to as “Variable Oxygen Coasting” or “Variable FiO₂ Coasting”.

A reduction in the amount of oxygen currently present in the line system may result from different situations, operating states of the anesthesia system, patient situations during the course of anesthesia or from effects from the operation of the anesthesia system or aspects of the integration of the anesthesia system into a data network (LAN, WLAN).

For example, situations can be determined on the basis of measured values for oxygen concentrations in the breathing gas or in the line system during the performance of anesthesia by an anesthesia system, situations can be determined from which a reduction in the oxygen concentration, which is provided to the patient, is necessary in the further course of the performance of the anesthesia. The measured values for oxygen concentrations in the breathing gas or in the line system can be recorded by means of an oxygen sensor system, for example in the form of an electrochemical, paramagnetic or semiconductor oxygen gas sensor, and made available to the control unit. The oxygen sensor system can be arranged in the anesthesia system, for example on the unit for mixing and dosing, inspiratory or expiratory on the circuit or line system or close to the patient on the Y-piece.

For example, user interactions, such as adjustments to the oxygen setpoint settings, may result in a need or a reason to reduce the amount of oxygen currently present in the line system. As a result of user interactions, for example after an O₂ flush function has been activated, there may subsequently be a need or reason to reduce the amount of oxygen currently present in the line system.

A verification with a result that there is a reason to reduce the amount of oxygen currently present in the line system results in the generation of an output signal that indicates this reason. The verification with generation and subsequent provision of the output signal can be carried out by the control unit. The output signal can be provided from the control unit to the mixing unit for mixing and dosing a gas mixture using data connections or data lines in the anesthesia system and/or via data networks (WLAN, LAN, Bluetooth), for example.

In a preferred embodiment, the supply of oxygen can be limited—preferably by the control unit—by reducing the amount of oxygen. The amount of oxygen can be reduced by influencing the control or monitoring of the mixing unit.

An increase (wash out) in the total quantity of fresh gas can be used as an action (a measure) to reduce the effective partial quantity of oxygen in the quantities of inhalation gas mixture supplied to the patient.

As an alternative or additional action to reduce the amount of oxygen, the gas mixture can be adjusted in such a way that, to reduce the oxygen concentration, the proportion of oxygen that is supplied into the fresh gas mixture (FG) via an oxygen gas inlet is reduced to zero, so that only a 21% proportion of oxygen, which is fed into the fresh gas mixture (FG) as part of the medical air (air) via an air gas inlet, causes a reduction in the oxygen concentration in the circuit system and for the patient after a certain period of time.

In a further preferred embodiment, the limitation of the supply of quantities of oxygen—preferably by the control unit—can be based on the oxygen concentration falling below (undershooting) a predetermined lower threshold value.

Falling below the predetermined lower threshold value of an oxygen concentration can be used to terminate the initiated action if this predetermined lower threshold value is not reached. An example of a lower threshold value is an oxygen concentration of 30 % in the breathing gas. Such a lower threshold value of the oxygen concentration can be derived from medical-physiological guidelines, such as those published and documented in the clinical guidelines of the medical associations for intensive care medicine and anesthesia.

In a further preferred embodiment, the limitation of the supply of quantities of oxygen can be configured using an error threshold around an absolute limit value or around a relative limit value of an inspiratory oxygen fraction (FiO₂) or an inspiratory oxygen concentration.

Absolute limit values or relative limit values of an inspiratory oxygen fraction (FiO₂) or an inspiratory oxygen concentration and error bands adapted to them can be derived from settings of the oxygen setpoint values and/or from medical-physiological specifications, such as those published and documented in clinical guidelines of the medical associations of intensive care medicine and anesthesia.

In a further preferred embodiment, a physiological limit situation can be taken into account (can be a basis) when configuring (defining) the predetermined lower threshold value.

Such a physiological limit situation can be derived from medical-physiological guidelines, such as those published and documented in the clinical guidelines of the medical associations for intensive care medicine and anesthesia.

Absolute limit values or relative limit values of an inspiratory oxygen fraction (FiO₂) or an inspiratory oxygen concentration as well as lower threshold values or the physiological limit situation can, in particular embodiments, also be derived from information or measured values of an oxygen saturation in the blood and/or from information of a blood gas analysis with regard to concentrations of oxygen and/or carbon dioxide.

In a further preferred embodiment, a patient category or a patient type can be included in the configuration of the predetermined lower threshold value or the absolute or relative limit value based on the physiological limit situation. Patient categories can be used to include individual tidal volumes and/or minute volumes, and thus advantageous and specific adjustments can be made when controlling the dosing quantities of oxygen with regard to patient categories such as adults, adolescents, children, infants as well as newborns or premature babies.

In a further preferred embodiment, hysteresis can be taken into account—preferably by the control unit—when limiting the supply of quantities of oxygen with the alignment at the predetermined lower threshold value of an oxygen concentration or the absolute or relative limit value of an oxygen concentration.

The advantage of hysteresis is that it prevents unwanted switching operations for additional doses of fresh gas when carrying out the actions described above, especially if the actual amount of oxygen in the breathing system and/or in the line system almost corresponds to the predetermined lower threshold value of an oxygen concentration.

In a further preferred embodiment, the supply of oxygen can be limited—preferably by the control unit—by reducing the amount of oxygen for a predetermined period of time (duration). The predetermined period of time can be based, for example, on typical durations resulting from physical relationships. Alternatively, the predetermined period of time can also be determined by means of experiments with reductions from a first level of an oxygen quantity to a second level of an oxygen quantity. For this purpose, time intervals for typical reductions, for example from 60% to 30%, from 80% to 60%, from 95% to 80%, from 30% to 21%, can be stored in the form of tables, which can then be selected accordingly, combined with each other if necessary and applied.

A device for carrying out the process according to the invention can have at least the following components:
a control unit,
a breathing gas conveying unit,
a sensor system for gas analysis, in particular for the analysis of oxygen,
a mixing unit for mixing and dosing a gas mixture of at least two gas components,
a line system consisting of hose lines for a fluidic connection with a patient,
   The control unit can be configured with the unit for mixing and dosing in order to carry out the process steps.

The device can also have at least one of the other components:
the flush valve assembly,
the APL valve assembly,
a manual ventilation bag (manual breathing bag),
a dosing unit for dosing volatile anesthetics,
an output unit for providing output signals,
an input interface for receiving input signals or user interactions.
   The device can form an anesthesia system with the components and/or the further components for performing anesthesia on a patient.

In the following, exemplary embodiments of the invention are explained in more detail with reference to the figures, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an anesthesia device; and

FIG. 2 is a flow sequence of a process for controlling gas quantities.

### DESCRIPTION OF PREFERRED EMBODIMENTS

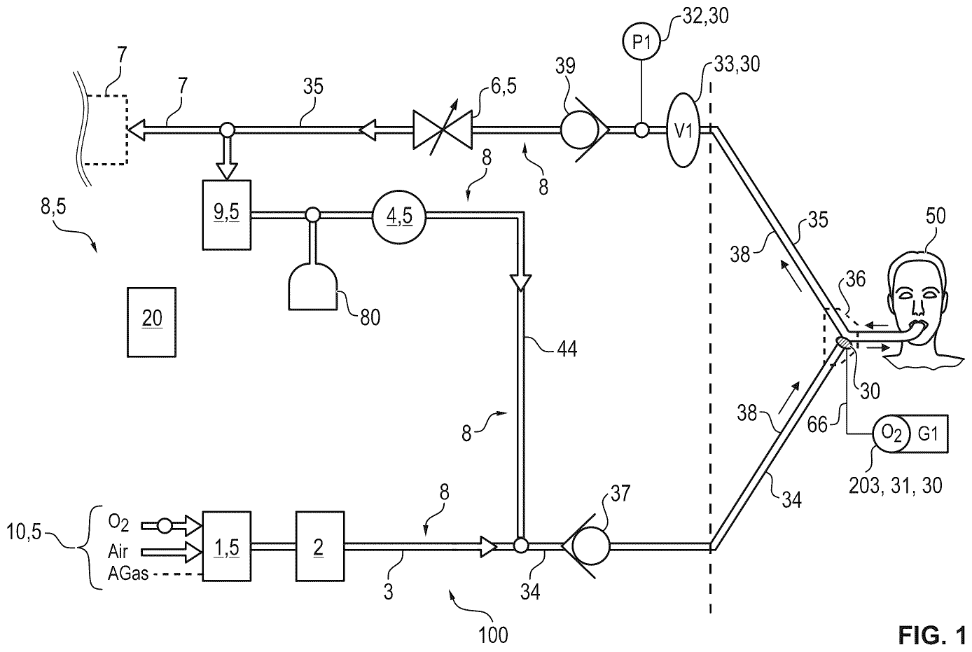

Referring to the drawings, FIG. 1 shows a basic structure and setup of an anesthesia device 100 with breathing gas supply system 5, with an anesthetic dosing unit (vaporizer) 2, a mixing unit 1, a gas conveying unit 4, an expiration valve 6, a circuit system 8 and an absorber unit 9. Gases such as oxygen, air or nitrous oxide are supplied to the mixing unit 1 via a gas supply 10. The absorber unit 9 enables the removal of quantities of carbon dioxide from the circuit system 8. The circuit system 8 has an inspiratory path 34 and an expiratory path 35 as well as an inspiratory non-return valve 37 and an expiratory non-return valve 39. The inspiratory and expiratory paths 34 and 35 are routed to a patient 50 via a connecting element (Y-piece) 36 via a line system 38, thus establishing the patient's pneumatic connection to the anesthesia device 100. The anesthesia device 100 has a sensor system 30 with at least one pressure sensor P1 32, at least one flow rate sensor V1 33 and at least one gas sensor G1 31 configured as an oxygen sensor. Further sensors 30 with alternative or additional pressure sensors, flow rate sensors or gas sensors can supplement the anesthesia device 100. An anesthetic gas scavenging device 7 may be present or connected to the circuit system 8.

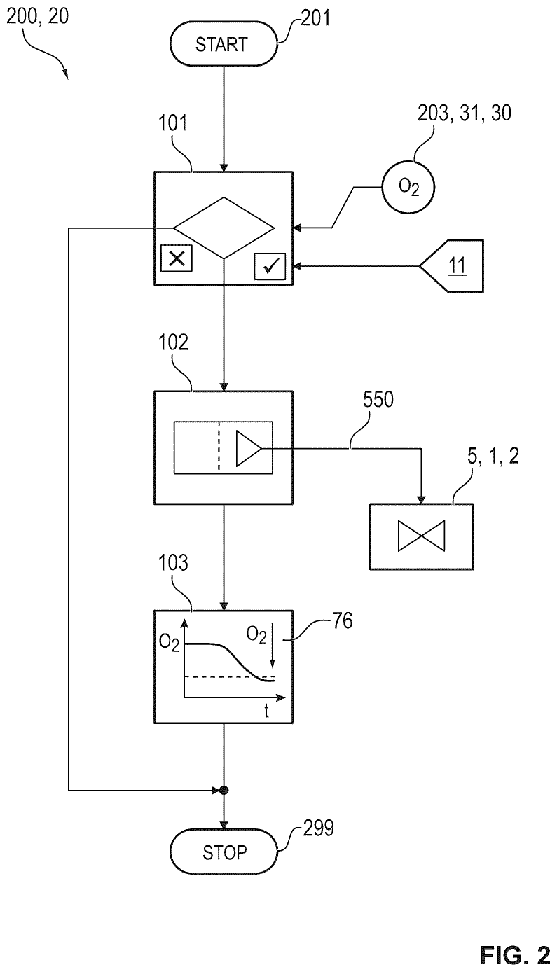

A control unit 20 is used to control the anesthesia device 100 and to carry out the process described in more detail in FIG. 2 for controlling gas quantities when dosing and supplying quantities of oxygen to an anesthesia system.

The control by the control unit 20 can be configured with aspects of coordination, sequence control, status control and/or regulation.

FIG. 1 shows a breathing bag BB 80 in an arrangement on the gas conveying unit 4; alternative arrangements of the breathing bag BB 80 on the circuit system 8 are possible. The at least one gas sensor G1 31 is configured as a near patient O₂ gas sensor 31 with an extraction (sampling) measurement functionality by means of a sample gas (measurement gas) line 66 for a measurement at the patient connecting element (Y-piece) 36. In optional embodiments, the at least one gas sensor 31 can also be arranged at other positions in or on the circuit system 8, for example downstream of the inspiratory non-return valve 37 in the direction of flow. The at least one pressure sensor P1 32 can also be arranged at other positions in or on the circuit system 8 or on the breathing gas supply system 5.

FIG. 2 shows a sequence of steps for carrying out a process for controlling gas quantities when dosing and supplying quantities of oxygen.

In a process 200, after a start 201 in a first step 101, a verification (a determination) is carried out as to whether there is a reason 11 for a reduction in a quantity of oxygen currently present in the line system.

In a second step 102, if there is a reason 11 for a reduction in the amount of oxygen currently present in the line system 38, an output signal 550 is generated, which is provided to the unit 5 for mixing and dosing a gas mixture. If there is no reason 11 for a reduction in the amount of oxygen currently present in the line system 38, the process comes directly to an end 299.

In a third step 103, a limitation 76 of the supply of quantities of oxygen into the line system 38 is effected, after which the process comes to an end 299.

While specific embodiments of the invention have been shown and described in detail to illustrate the application of the principles of the invention, it will be understood that the invention may be embodied otherwise without departing from such principles.

### Reference Number List

1 Mixing unit
2 Anesthetic dosing unit
3 Fresh gas line
4 Gas conveying unit
5 Breathing gas supply system
6 Expiratory dosing device, expiratory valve
7 Anesthetic gas supply unit, AGS (AGSS—anesthetic has scavenging system)
8 Circuit system
9 Absorber unit, CO₂ absorber
10 Gas supply
11 Reason
20 Control unit
30 Sensors
31 O² gas sensor G1
32 Pressure sensor P1
33 Flow rate sensor V1
34, 35 Inspiratory path, expiratory path
36 Connecting element, Y-piece,
37, 39 Non-return valves, inspiratory and expiratory
38 Line system, breathing circuit
44 Rebreathing circuit
50 Living being, patient
66 Sample gas line
76 Limiting the amount of oxygen
80 Breathing bag BB
100 Anesthesia device, anesthesia system
101, 102, 103 Step sequence
200 Process
201 Start
203 Measured value acquisition by the control unit
299 End, stop
550 Output signal

What is claimed is:

1. A process for controlling gas quantities when dosing and supplying quantities of oxygen in a line system by means of a mixing unit for mixing and dosing a gas mixture of breathing gas of at least two gas components, the process comprising the steps of:
in a first step carrying out a verification as to whether there is a reason for a reduction in an amount of oxygen currently present in the line system or in the breathing gas;
in a second step, if it is verified that there is a reason for a reduction in an amount of oxygen currently present in the line system, generating an output signal, which indicates this reason and providing the generated output signal to the mixing unit for mixing and dosing the gas mixture; and
in a third step, effecting a limitation of a supply of quantities of oxygen into the line system, based on the generated output signal.

2. A process according to claim 1, wherein the limitation of the supply of quantities of oxygen into the line system comprises reducing an amount of oxygen dosed and supplied.

3. A process according to claim 2, wherein reducing an amount of oxygen dosed and supplied comprises undershooting a predetermined lower threshold value of an oxygen concentration.

4. A process according to claim 3, wherein a physiological limit situation is taken into account in defining the predetermined lower threshold value.

5. A process according to claim 4, wherein the physiological limit situation is based on a patient category or is based on a patient type and is taken into account in defining the predetermined lower threshold value.

6. A process according to claim 3, wherein hysteresis is taken into account in defining the predetermined lower threshold value.

7. A process according to claim 2, reducing an amount of oxygen dosed and supplied is configured based on an error band around an absolute limit value or around a relative limit value of an inspiratory oxygen fraction or of an inspiratory oxygen concentration.

8. A process according to claim 7, wherein a physiological limit situation based on a patient category or a physiological limit situation based on a patient type is taken into account in the configuration of the absolute limit value or the relative limit value.

9. A process according to claim 7, wherein hysteresis is taken into account in the configuration of the absolute or relative limit value.

10. A process according to claim 2, wherein reducing an amount of oxygen dosed and supplied comprises reducing the quantity of oxygen for a predetermined period of time.

11. An anesthesia system comprising:
a line system configured to guide a beathing gas for a fluidic connection to a patient;
a mixing unit connected to the line system, the mixing unit being configured to mix and dose a gas mixture of at least two gas components, including quantities of oxygen, into the line system;
a sensor system for gas analysis, the sensor system being operatively connected to the line system; and
a control unit operatively connected to the sensor system and operatively connected to the mixing unit, the control unit being configured to:
control the mixing unit for mixing and dosing of the gas mixture into the line system;
verify whether there is a reason for reducing an amount of oxygen currently present in the line system or in the breathing gas;
generate an output signal upon verifying the reason for reducing an amount of oxygen currently present in the line system, the output signal comprising an indication of the reason, and output the generated output signal to the mixing unit; and
control a limiting of oxygen supplied into the line system.

12. An anesthesia system according to claim 11, further comprising a circuit system connected to the line system, wherein the mixing unit is connected to the line system via the circuit system, the mixing unit being configured to mix and dose the gas mixture of at least two gas components, including quantities of oxygen, into the circuit system wherein the mixing device is controllable to selectively mix and dose medical air from a medical air source and oxygen from an oxygen source into the circuit system.

13. An anesthesia system according to claim 11, further comprising at least one of:
a breathing gas conveying unit;
a manual ventilation bag;
an output unit;
a dosing unit for dosing volatile anesthetics;
an input interface for receiving input signals or user interactions;
an output interface for providing output signals.

14. A process for controlling gas quantities supplied by an anesthesia system, the process comprising the steps of:
providing an anesthesia system, which comprises: a line system configured to guide a beathing gas for a fluidic connection to a patient; a circuit system connected to the line system; a mixing unit connected to the line system via the circuit system, the mixing unit being configured to mix and dose a gas mixture of at least two gas components, including quantities of oxygen, into the circuit system, wherein the mixing device is controllable to selectively mix and dose medical air from a medical air source and oxygen from an oxygen source into the circuit system; a sensor system for gas analysis, the sensor system being operatively connected to the line system and/or the circuit system; and a control unit operatively connected to the sensor system and operatively connected to the mixing unit;
with the control unit, controlling the mixing unit for mixing and dosing of the gas mixture into the line system;
with the control unit, verifying whether there is a reason for reducing an amount of oxygen currently present in the line system or in the breathing gas;
with the control unit, generating an output signal upon verifying the reason for reducing an amount of oxygen currently present in the line system, the output signal comprising an indication of the reason, the output signal being output to the mixing unit; and
with the control unit, controlling a limiting of oxygen supplied into the circuit system.

15. A process according to claim 14, wherein the limiting of oxygen supplied comprises reducing an amount of oxygen dosed and supplied from the oxygen source into the circuit system.

16. A process according to claim 15, wherein reducing an amount of oxygen dosed and supplied comprises undershooting a predetermined lower threshold value of an oxygen concentration.

17. A process according to claim 16, wherein the predetermined lower threshold value is defined based on a physiological limit situation.

18. A process according to claim 16, wherein the predetermined lower threshold value is defined based on hysteresis of the anesthesia system.

19. A process according to claim 14, reducing an amount of oxygen dosed and supplied is configured based on an error band around an absolute limit value or around a relative limit value of an inspiratory oxygen fraction or of an inspiratory oxygen concentration.

20. A process according to claim 14, wherein limiting of oxygen supplied comprises reducing an amount of oxygen dosed and supplied for a predetermined period of time.
